(19) United States
(12) Patent Application Publication
(10) Pub. No.: US 2026/0337694 A1
(43) Pub. Date: Oct. 29, 2026

(54) TUMOR FRACTION ESTIMATION

(71) Applicant: CLEARNOTE HEALTH, INC., San Diego, CA (US)

(72) Inventors: Samuel Levy, San Diego, CA (US); Wayne Volkmuth, San Diego, CA (US); Onur Sakarya, San Diego, CA (US); Gulfem Guler, San Diego, CA (US)

(21) Appl. No.: 19/640,579

(22) Filed: Apr. 7, 2026

Related U.S. Application Data
(60) Provisional application No. 63/784,658, filed on Apr. 7, 2025.

(51) Int. Cl. C12Q 1/6886; C12Q 1/6869; G06N 20/00; G16B 20/10; G16B 30/00; G16B 40/00

(52) U.S. Cl. CPC C12Q 1/6886; C12Q 1/6869; G16B 20/10; G16B 30/00; G16B 40/00; C12Q 2600/156; G06N 20/00

(57) ABSTRACT

The disclosure provides methods of estimating the tumor fraction in a biological sample. In some embodiments, the methods may be implemented by a machine learning tool trained on sequence data from at least tumor tissue and healthy tissue. In some embodiments, the machine learning system is trained on residual and/or normalized genomic read counts corresponding to known tumor fractions.

### FIELD OF THE INVENTION

The invention generally relates to a method of estimating the tumor fraction of a biological sample.

### BACKGROUND

One of the primary challenges in cancer diagnostics and treatment is the ability to monitor disease progression and therapy response effectively. Cell-free DNA (cfDNA) contains DNA released from various cells in the body, including tumor cells, and accurately quantifying the tumor fraction in cfDNA can be helpful since tumor cfDNA can vary depending on the cancer stage, size, and location of the tumor. Unfortunately, accurately quantifying the tumor fraction in cfDNA is also challenging, especially when the tumor fraction is low.

Accurately measuring the change in this quantity over time can provide valuable insights into how a cancer is progressing and how well it is responding to treatment. For example, cancers that respond to therapy typically have lower fractions of tumor cfDNA relative to a baseline state.

Higher tumor fraction also indicates poor prognosis in many cancer types. Measurement of tumor fraction can also help stratify patients in high or low risk groups, and aid in identification of patients who are more likely to relapse after treatment and therefore benefit from additional therapies.

Conventional methods for estimating tumor fraction have limitations. For example, existing methods, such as ichorCNA, have tumor fraction detection thresholds of between 3-10%, and some reports indicate inaccurate results below 20%. Further, since ichorCNA is based on copy number variation (CNV), it may not be accurate for cancers that are not driven by CNVs. Other methods, such as VAF-based ctDNA fraction estimation are constrained by assumptions about clonality, ploidy, and copy number, making them sensitive to subclonal mutations and genomic alterations that can bias the inferred tumor fraction and lead to inaccurate results.

Previous work on prostate cancer has demonstrated that 5hmC patterns are specific for prostate cancer-derived DNA, enabling the accurate quantification of circulating tumor fraction. Accordingly, there is a need for expanded methods to estimate tumor fraction from 5hmC samples for a wider range of samples and cancer types. Further, there is also a need for methods to more accurately estimate the fraction of cell-free DNA (cfDNA) derived from tumor cells in a biological sample (e.g., a blood sample).

In summary, there is a critical need in cancer diagnostics to provide a more accurate and reliable method for estimating tumor fraction in cfDNA, particularly while using 5hmC data.

### SUMMARY

The present invention provides more accurate and reliable measurements of tumor fraction using epigenetic data. Accordingly, a method of estimating tumor fraction in a sample is disclosed. In a preferred embodiment, the epigenetic data are 5-hydroxymethylcytosine (5hmC) derived data. The invention is applicable to any epigenetic markers, however. In contrast to CNV-based methods, 5-hydroxymethylcytosine (5hmC) signals are related to gene expression and gene regulation, and they may still be able to detect tumor fraction accurately in cases with lower or no level of CNV. Accordingly, one method of providing more accurate and reliable measurements of tumor fraction is using 5hmC data. DNA containing 5hmC is enriched at active or poised to be active regions of the genome (e.g., transcriptionally active gene bodies, the borders of active promoters and enhancers, repetitive elements etc.) and has been implicated in cancer development and progression. Some 5hmC signals may be shared between cancers of different types or origin tissues, while others may be specific to a type of cancer or a tissue of origin. Therefore, accurately measuring tumor fraction from 5hmC signals may require a cancer type-specific strategy.

In some embodiments, methods comprise obtaining cfDNA from a biological sample, and using a portion of the cfDNA obtained, separating cfDNA fragments containing epigenetic markers. The separated cfDNA is sequenced to generate a first set of genomic sequences. The cfDNA remaining from the sample not used in the separating step is also sequenced to generate a second set of genomic sequences. The first and second set of genomic sequences are mapped to reference sequences. Counts of mapped sequences are normalized to generate normalized first genomic sequence counts and normalized second genomic sequence counts. Additionally, the differences between the second and first sets of genomic sequences—referred to as ‘residual genomic sequence counts’—are computed. These residual counts reflect epigenetically driven biological signals after accounting for variations caused by copy number changes. The fraction of the cfDNA that is tumor derived is estimated based on the number of residual and/or normalized genomic sequence counts.

Some of the disclosed methods operate a machine learning system to estimate the tumor fraction. The machine learning system may include a processor and a memory including a non-transitory computer readable medium for storing, reading, and processing data such as computer instructions or code, sequence data including FASTA, FASTQ, BED, VCF, AB1, SAM, and BAM files, and calculations for analyzing the sequence data and estimating a tumor fraction.

In some embodiments, the machine learning system is trained on sequence data from tumor samples and healthy samples. The sequence data may be whole genome sequencing reads. In some embodiments, the machine learning system is trained on residual genomic read counts and/or normalized genomic read counts of samples with known tumor fractions. Some embodiments utilize a machine learning input comprising both residuals and FPKM values. The machine learning system may also comprise a regularized linear regression classifier.

For some methods of the invention, the normalized modified read counts comprise fragments per kilobase per million fragments (FPKM). Further, in some embodiments, the FPKM are log-transformed.

Some methods of the disclosure include building the machine learning system by sequencing nucleic acid from samples with known tumor fractions to obtain genomic reads. Portions of the nucleic acid comprising modified bases are treated and then sequenced to obtain modified reads. The genomic reads and the modified reads are mapped to reference data. Counts of mapped reads may be normalized to provide normalized genomic read counts and normalized modified read counts. The effects of copy number variation (CNV) are removed from the normalized genomic read counts and the result is saved as a residual. Finally, the machine learning system is trained using at least the residual and/or the normalized genomic read counts, and the known tumor fractions. The machine learning system outputs an estimated tumor fraction when given the input comprising at least the subject residual or normalized genomic read counts.

In some aspects of the disclosure, methods of estimating tumor fraction in a sample include separating a first portion of cfDNA from a biological sample of cfDNA fragments containing epigenetic markers, sequencing the separated first portion of cfDNA to generate a first set of genomic sequences, sequencing cfDNA remaining in the sample after the separating step to generate a second set of genomic sequences, mapping the first and second set of genomic sequences to reference sequences, normalizing counts of mapped sequences to generate normalized first genomic sequence counts and second genomic sequence counts, identifying repeated genomic insertions and/or deletions by measuring the depth of sequence read coverage from the second set of genomic sequences, subtracting the repeated insertions and/or deletions identified in the identifying step from the first set of genomic sequences to determine a number of residual genomic sequence counts, and estimating the tumor fraction as a fraction of the cfDNA that is tumor derived based on the number of residual and/or normalized genomic sequence counts. Methods may include preparing a sequence library from the biological sample.

In some embodiments, the normalized read counts include fragments per kilobase per million fragments (FPKM). The FPKM may be log-transformed. The cancer in which the tumor fraction is being measured may include breast, colorectal, gastric, esophagus, liver, lung, ovarian, pancreatic cancer, and prostate cancer. The epigenetic marker may include at least one of 5hmC, CEACAM5, SYP, SRRM4, AR, CHGA, CELF3, LMO3, SNAP25, CHRNB2, PCSK1, NEUROD1, ALDH1A3, HOMER2, TSC22D1, ELL2, STEAP4, TARP, and DNAJB9. In certain embodiments, either or both genomic sequence counts may be normalized using SVD methodology.

Preferred methods may include operating a machine learning system trained on sequence data from at least tumor tissue/cfDNA, and healthy tissue/cfDNA. The input to the machine learning system comprises concatenation of residuals, FPKM values, fragmentomics or other fragment properties. The machine learning system may include a regularized linear regression classifier, and other non-linear classifiers.

In some embodiments, the machine learning system may be built by sequencing nucleic acid from samples with known tumor fractions to obtain genomic reads, treating and sequencing portions of the nucleic acid comprising modified bases to obtain modified reads, mapping the genomic reads and the modified reads to reference data, normalizing counts of mapped reads to provide normalized genomic read counts and normalized modified read counts, removing effects of copy number variation (CNV) from the normalized genomic read counts and saving the result as a residual, wherein the residual comprises normalized modified read counts corrected to remove the effects of the CNV, and training the machine learning system using at least the residual, the normalized genomic read counts, and the known tumor fractions to output an estimated tumor fraction when given the input comprising at least the subject residual or normalized genomic read counts.

In certain aspects of the disclosure, a machine learning system for estimating a tumor fraction in a sample may include at least one processor coupled to a memory subsystem having instructions stored therein executable by the processor to cause the system to: receive a first set of genomic sequences, the first set of genomic sequences sequenced from a portion of a biological sample of cfDNA fragments containing epigenetic markers, receive a second set of genomic sequences, the second set of genomic sequences sequenced from a remaining portion of the biological sample, map the first and second set of genomic sequences to reference sequences, normalize counts of the mapped sequences to generate a normalized first genomic sequence count and a normalized second genomic sequence count, identify repeated genomic insertions and/or deletions by measuring a depth of sequence read coverage from the second set of genomic sequences, subtract the repeated insertions and/or deletions identified in the identifying step from the first set of genomic sequences to determine a number of residual genomic sequence counts, and estimate a tumor fraction as a fraction of the cfDNA that is tumor derived based on the number of residual and/or normalized genomic sequence counts.

In some embodiments, the system is trained on sequence data from at least tumor tissue/cfDNA, and healthy tissue/cfDNA. The input to the system may include concatenation of residuals, the FPKM values, fragmentomics or other fragment properties, The system may be trained using ridge regression, LASSO, elastic net regression, adaptive LASSO, fused LASSO, dimensionality reduction, the ensemble method, feature selection methods, Bayesian regression, robust regression, or generalized least squares methods.

In certain embodiments, the system may be built by sequencing nucleic acid from samples with known tumor fractions to obtain genomic reads, treating and sequencing portions of the nucleic acid comprising modified bases to obtain modified reads, mapping the genomic reads and the modified reads to reference data, normalizing counts of mapped reads to provide normalized genomic read counts and normalized modified read counts, removing effects of copy number variation (CNV) from the normalized genomic read counts and saving the result as a residual, wherein the residual comprises normalized modified read counts corrected to remove the effects of the CNV, and training the machine learning system using at least the residual, the normalized genomic read counts, and the known tumor fractions to output an estimated tumor fraction when given the input comprising at least the subject residual or normalized genomic read counts.

The machine learning system may be trained to measure a tumor fraction for breast, colorectal, gastric, esophagus, liver, lung, ovarian, pancreatic cancer, and prostate cancer. The epigenetic marker of the input data may include at least one of 5hmC, CEACAM5, SYP, SRRM4, AR, CHGA, CELF3, LMO3, SNAP25, CHRNB2, PCSK1, NEUROD1, ALDH1A3, HOMER2, TSC22D1, ELL2, STEAP4, TARP, and DNAJB9. In certain embodiments, either or both genomic sequence counts may be normalized. The normalization may be achieved using SVD methodology.

### BRIEF DESCRIPTION OF THE DRAWINGS

FIG. 1 illustrates training data according to one or more embodiments of the invention.

FIG. 2 shows the genome-wide distribution of 5hmC coefficients according to one or more embodiments of the invention.

FIG. 3 shows genome-wide distribution of 5hmC coefficients after CNV filtering according to one or more embodiments of the invention.

FIG. 4 illustrates a cross-validation procedure according to one or more embodiments of the invention.

FIG. 5 shows cross-validation results according to one or more embodiments of the invention.

FIG. 6 shows cross-validated test predictions vs expected TF according to one or more embodiments of the invention.

FIG. 7 shows a 3-way comparison of 5hmC, ichorCNA vs targeted mutation-based TF (%) estimates according to one or more embodiments of the invention.

FIG. 8 shows an ichorCNA genome-wide plot of outlier samples according to one or more embodiments of the invention.

FIG. 9 shows an in-silico simulation performance of TF estimates according to one or more embodiments of the invention.

FIG. 10 shows an in-silico simulated TF distribution according to one or more embodiments of the invention.

FIG. 11 shows Kaplan-Meier survival curves for TF low vs high groups according to one or more embodiments of the invention.

FIG. 12 shows a heatmap of the top 1000 TF-associated gene features according to one or more embodiments of the invention.

FIG. 13 shows Kaplan-Meier survival curves for TF low vs high groups according to one or more embodiments of the invention.

FIG. 14 shows gene set enrichment analysis of a 5hmC model.

FIG. 15 shows gene set enrichment analysis of a m0 rank order of 5hmC model features demonstrating enrichment of an androgen response geneset and increased enrichment over an androgen response geneset with increasing ct fraction according to one or more embodiments of the invention.

FIG. 16 shows plasma-derived cfDNA 5hmC profiles can distinguish between AR-specific and neuroendocrine-specific biology in prostate cancer according to one or more embodiments of the invention.

FIG. 17 shows SVD normalization reduces GC-dependent bias in 5hmC data according to one or more embodiments of the invention.

FIG. 18 shows a Limit of blank (LoB) reduction by adding WGS fragmentation features, SVD normalization and weighting in glmnet modeling according to one or more embodiments of the invention.

FIG. 19 shows a Limit of Blank (LoB) and Limit of Detection of an embodiment of a model for estimating ct fraction according to the disclosure.

### DETAILED DESCRIPTION

Methods of the invention have significantly improved prediction of tumor fraction using 5hmC features by incorporating tissue and/or cfDNA samples into the training phase of an algorithm. In some embodiments, the disclosed methods include non-cancer cfDNA samples to teach the model based on samples with zero tumor fraction. Consequently, the training samples utilized as part of the present disclosure are larger, especially with the inclusion of non-cancer samples, and further expansion of the tumor tissue/cfDNA cancer dataset.

The disclosed methods also include more complex machine learning features as predictors. Some specific features, referred to as residual and concatenated feature sets, are explained further below. The disclosed model is trained on and operates on samples collected in, for example, Streck or EDTA tubes.

The present disclosure also contemplates cancer subtype specific models. Specifically, the disclosed methods may or may not include a specific subtype of prostate cancer called neuroendocrine prostate cancer in the training sets. In the present disclosure, including or not including that subtype yielded differences in the resulting model. Accordingly, the invention contemplates advanced models that take into account cancer subtype to improve the accuracy of a tumor fraction estimate. In some alternatives, specific details in the way the model is trained (using inner and outer cross validation) provide further differentiation.

In some embodiments, a model according to the invention predicts tumor fraction with equivalent or better performance than conventional methods, and with higher specificity than, for example, ichorCNA. The examples below provide several comparisons with ichorCNA.

Some methods of the disclosure provide an accurate and reliable tumor fraction estimation. Some methods of the disclosure provide estimations that can be used for minimal residual disease detection, prognostication, patient stratification, therapy response monitoring, improved biomarker analysis, and potentially improved cancer prediction.

The disclosed methods, compared to mutation or copy-number based tumor fraction approaches, are more reliable, especially when the samples are lacking any known driver mutations or contain few copy-number alterations. It is known that about 5% of tumors are missing driver mutations and that about 90% of tumors contain copy number alterations, but some tumor types have more stable genomes and do not show significant aneuploidies (gain or loss of entire chromosome or chromosome arms), including prostate and some types of pancreatic cancers. Furthermore, some tumors are driven only by translocation/gene fusion mutations (e.g. BCR-ABL fusion driven cancers).

In theory, a tumor with low copy number alterations is more challenging to detect with ichorCNA, particularly when tumor fraction is low (e.g. 3% to 20% range). In contrast, 5hmC-based approaches leverage genome-wide epigenetic patterns that are less dependent on discrete mutations, enabling more robust and more accurate estimation of tumor contribution, especially in heterogeneous or low-mutation contexts. In fact, in one exemplary analysis, tumor fraction was predicted to be about 20% with both targeted mutation and the 5hmC approach of the invention but was not detected by ichorCNA (0% tumor fraction). There have also been cases in which the specificity of ichorCNA calls were questionable. For example, 3 out of 224 non-cancer ITTP samples had predictions of ichorCNA tumor fraction in a 10% to 20% range, whereas 5hmC predictions were, as expected, around 0% in these cases. Utilizing the disclosed 5hmC based tumor fraction methods in these cases resulted in no false positives.

In some embodiments, a library is generated using cfDNA from a biological sample. The cfDNA from the sample may be prepared by PCR by, for example, amplifying adaptor ligated fragments in the sample. The library is sequenced to generate sequences, and the sequences may be mapped to reference data to identify locations within the sample nucleic acid (and patterns of the locations) that contain the epigenetic modifications. To identify tissue variant genes, the raw counts of each gene feature may be normalized as counts per million (CPM) reads in each sample, and the normalized CPM may be Log 2 transformed.

For example, extracted or purified nucleic acid may be subject to any combination of fragmentation, end repair, A-tailing, and adaptor ligation. Some embodiments include fragmentation (e.g., enzymatically or mechanically) to a predetermined average fragment size and ligation to sequencing adaptors to yield adaptor ligated fragments. Thus, in preferred embodiments, library preparation steps may provide adaptor-ligated fragments of cfDNA from a liquid biopsy sample among which may be adaptor-ligated fragments of circulating-tumor DNA (ctDNA).

One potential application of the present invention is to use a trained model to improve tumor-fraction estimation. In some embodiments, a model is trained with samples from a specific cancer type, and that model is then used only with samples for that given cancer type. While the examples herein analyze prostate cancer, additional models for other cancers are also envisioned and would be apparent to the skilled artisan based on the present disclosure. It is clear that the invention is applicable to a pan-cancer model as well.

Improved tumor fraction estimates obtained using the disclosed methods are useful for minimal residual disease detection, where the goal is to determine (after treatment) the likelihood of a potential recurrence. The tumor fraction estimates are also incorporated as a feature of machine learning models that are used to detect cancer.

Measurement of tumor fraction estimates allows adjustment of 5hmC feature count (CPM, FPKM, etc.) so that these adjusted values are useful to train a prediction model for the detection of cancer or cancer type. More accurate measurements of tumor fraction allow such “normalization” of 5hmC feature counts so that cancer signals across populations, such as from multiple samples, are learned.

Additionally, model performance is enhanced by incorporating fragmentomic and epigenomic descriptors such as regional fragment size distributions, end-motif frequencies, and locus-specific hydroxymethylation signals. Applying robust normalization strategies—e.g., single value decomposition (SVD) normalization, reduces technical variance and stabilizes coefficient estimation in the glmnet framework. Furthermore, implementing a feature weighting scheme—such as inverse-variance weighting, prior-informed penalties, or adaptive lasso weights—can preferentially emphasize high-signal regions while suppressing noise-prone features. Collectively, these modifications improve predictive accuracy, as reflected by increased R², reduced MAE, and a lower limit of blank, thereby enhancing sensitivity in low ctDNA fraction regimes.

### Example 1

A set of tissue and cfDNA prostate cancer samples with tumor fraction estimated using copy-number (using a tool called ABSOLUTE for the tissue samples, and ichorCNA for cfDNA samples), was taken and added to a set of cfDNA samples from non-cancer subjects. Samples collected in both EDTA and Streck tubes were analyzed as the collection tube could in principle affect the recovery of cfDNA. The density of 5hmC (fragments per kilobase per million mapped reads, FPKM) in genes was used as one of the features for machine learning.

A subset of genes that were candidates for predicting tumor fraction were identified as potential features for the machine learning system. In this step, for each gene in the genome, a linear regression model of tumor fraction vs the gene's 5hmC density was built. Genes with significant p-values (after multiple test correction) were selected for the model building step.

Using the selected features, an elastic net linear regression was employed to build a tumor fraction prediction model. The model was built using an extra outer cross-validation framework to optimize hyper-parameters such as the elastic net alpha parameter, number of features selected, and the specific feature sets used for the model.

Both 5hmC and whole genome sequencing (WGS) features were used in modeling. Different models were constructed by transforming and combining the features in different ways. The transformations include log transformed 5hmC FPKM values alone (m0), log transformed WGS FPKM values alone (m3), the residual of the log 5hmC FPKM values from their prediction by log WGS FPKM values (m1), concatenated residual 5hmC and log FPKM WGS features (m2), and concatenated log transformed 5hmC FPKM values concatenated with log FPKM WGS features (m4). Residual 5hmC were obtained by a linear fitting of 5hmC vs WGS per gene. Better results overall were observed with the m2 feature set.

### Example 2

### Training and Validation Dataset

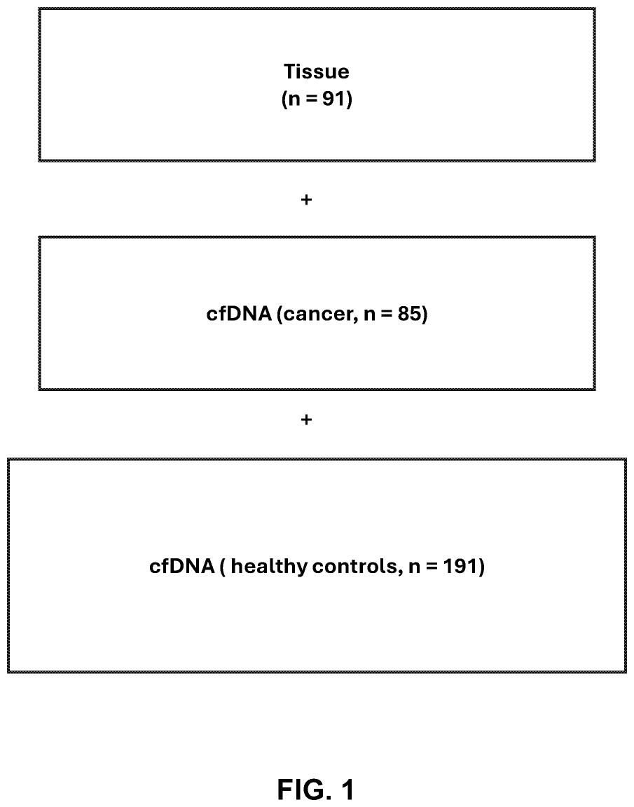

Tumor fraction prediction models were trained with 5hmC and low-pass WGS sequencing. As shown in FIG. 1, the training data is compiled from a metastatic castration-resistant prostate cancer (mCRPC) cohort with both tissue and cfDNA samples, and age-matched male healthy controls cfDNA samples, to give a good dynamic range of tumor fractions. Samples annotated as belonging to neuroendocrine positive or double negative subtypes (based on RNA expression in tumors) were not used. 5hmC-based features were trained to predict tumor fraction estimates from ABSOLUTE and ichorCNA using WGS data.

In certain embodiments, the training process for the model may include changes to sample weightings, alternative penalty functions, and two-stage models for predicting a binary outcome of 0 vs not 0 and estimating a specific value for not 0 cases. Two-stage models include a forecasting error or residuals using autoregressive integrated moving average (ARIMA), unobserved components model (UCM), or equivalent techniques, and applying the forecasting error to a linear regression model, such as those described below, to identify improvements in the model during training. The improved model may be selected for further training and revision. Two-stage models may include zero-inflated or hurdle models including a first stage of modeling the probability of a value being 0 or not 0 (binary classification) and a second stage determining the value of the not 0 outcomes, for example by applying a regression model. A one-stage model, such as a regression model, may be used to analyze the dataset in a single step using an appropriate model accounting for clustering within the dataset.

### Models and Features

Models may be trained using ridge regression, LASSO, elastic net regression, adaptive LASSO, fused LASSO, dimensionality reduction, the ensemble method, feature selection methods, Bayesian regression, robust regression, or generalized least squares methods. In some embodiments, data massaging techniques such as the use of a panel of normals and SVD normalization or analogous methods may be used to reduce batch effects and technical artifacts such as GC bias. GC bias occurs in high-throughput sequencing when DNA fragments are mis-represented based on guanine-cytosine content leading to uneven read coverage. For example, the Genome Analysis Toolkit (GALK) structured programming framework may be applied to analyze high-throughput sequencing data for identifying variants. Other examples may include the baseline estimation and denoising with sparsity (BEADS) correction model.

Base features used in the models included FPKM normalized log-5hmC read counts across each gene body (n=18.5k) along with FPKM normalized log-WGS read counts to represent copy number status. It was observed that 5hmC enriched reads contain matched CNV patterns in WGS data.

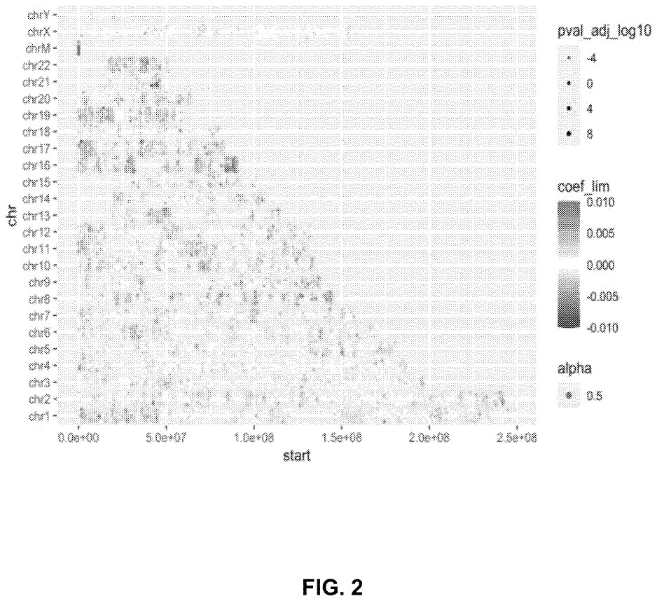

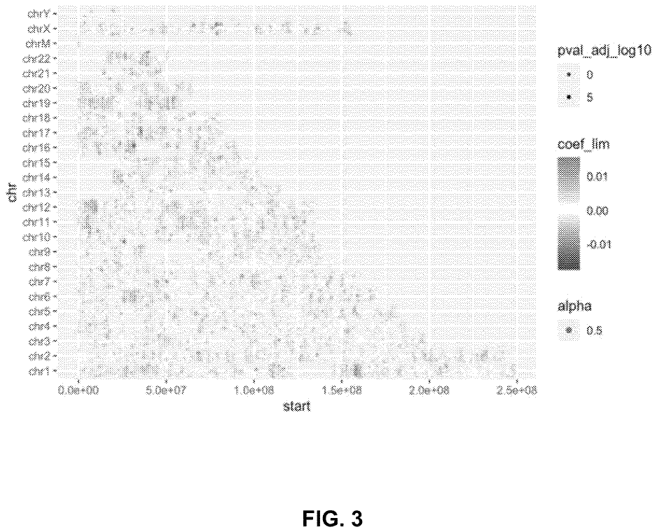

Residual 5hmC was added as a new feature type, in which copy number effect were removed from each of the 5hmC features. This was accomplished by taking the residual of the 5hmC counts from a linear fit to the WGS counts for each gene. These residual features captured 5hmC read count that was independent of the genomic copy number effect, thus focusing on the variation due to other factors. log-WGS features were also kept as a feature by concatenating them with residual 5hmC to form the final feature matrix for the model. On the genome-wide views shown in FIGS. 2 and 3, the effect of taking the residual for the 5hmC features is illustrated. FIG. 2 shows the coefficients of each gene versus tumor fraction based on 5hmC features, and FIG. 3 shows the coefficients with the CNV effect removed (based on WGS read counts).

In some embodiments, the residual approach may be applied to features of interest such as genes, promoters, enhancers, quantile bins, or other window approaches for dividing up the genome. Fragmentomics style features may be identified for segmenting windows such as overall length distribution of fragments, fragment end profiles, a fraction of fragments that are less than a defined length for various intervals along the genome, or a ratio of fragments in one length range to fragments in another length range for various intervals along the genome. A copy number identifying features such as an estimate of the difference in depth observed in a set of regions may be used to identify windows that are amplified or deleted.

For example, the genome may be tiled into quartile bins, windows of variable width that, in a baseline set of samples, contain equal proportions of the observed fragments. These windows may be of fixed width of, for example, 1 kilobase (kb), 2 kb, 5 kb, 10 kb, 25 kb, 50 kb, 100 kb, 250 kb, 500 kb, 750 kb, or 1 Megabase (Mb).

In certain embodiments, the windows are differential bins, determined by finding regions of over or under-representation in tumor samples versus control samples. These regions may be identified using segmentation methods such as computer-based systems (CBS), shifting level models (SLM), hidden Markov model (HMM), or other dynamic programming approaches known in the art.

### Hyper-Parameter Optimization

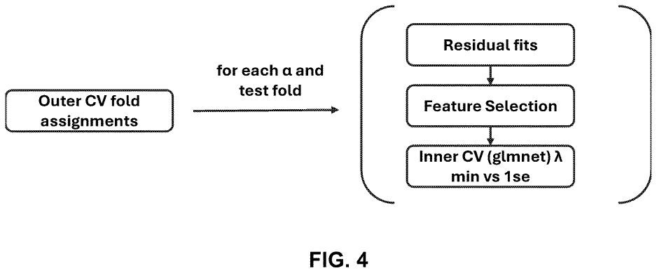

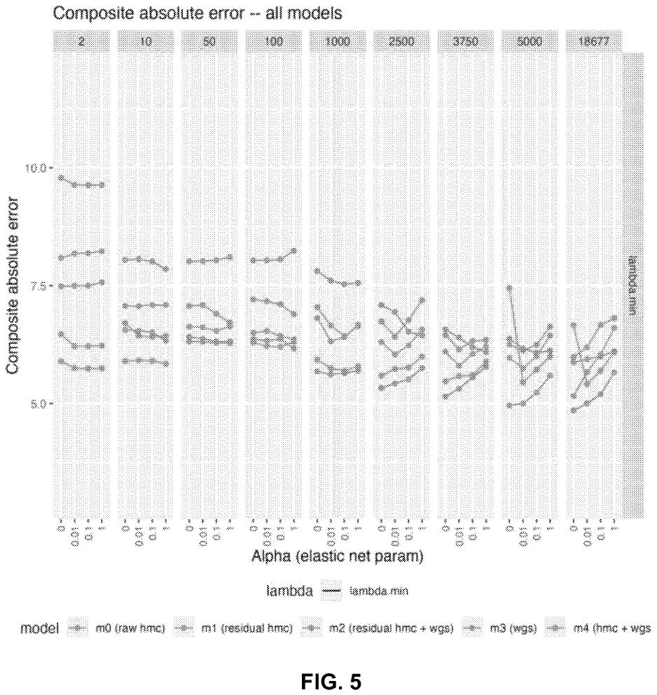

In some embodiments, models were trained using regularized linear regression package glmnet. Cross-validation within glmnet was used to select λ (regularization factor). As shown in FIGS. 4 and 5, an outer cross-validation was implemented to test hyper-parameters such as elastic net parameter α, number of features passed on to glmnet (facets in the plot), and model to use (m0 to m4, colored lines).

Features were selected to pass to glmnet based on the p-value of the slope of a linear regression fit for each gene vs the tumor fraction. Composite absolute error (CAE), defined as the average mean absolute error and median absolute error was used to assess performance. Final hyper-parameters were selected by combined error metrics across 5 shuffles (outer CV fold assignments).

In the cross-validation results plot of FIG. 5, only lambda-min was shown as it had better performance than lamda-1se overall. Samples from the same patient (tissue or cfDNA, baseline or progression) were kept in the same fold. Overall best performance was observed for the m2 model when all gene features were passed on to glmnet with alpha=0 (ridge regression).

### Training Set CV Achieves <5% Composite Absolute Error on a Diverse Sample Set

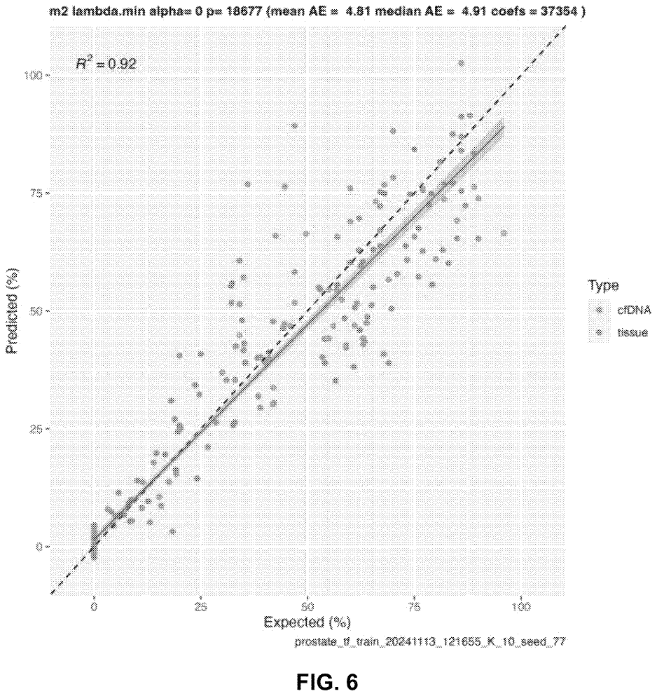

During cross validation (10 folds) phase of model training, samples left out in test sets (individual dots on the plot) were used to assess performance. Here (as shown in FIG. 6) the performance for cancer (expected >0%) and non-cancer (expected=0%) cases combined is illustrated, with colors showing sample type. The model predicted either sample type well. In FIG. 6, note that the expected % values (x-axis) are the tumor fraction estimates from CNV methods (ABSOLUTE and ichorCNA), while the y-axis shows the 5hmC-based estimates. The dotted line shows the line y=x.

5hmC-Based Tumor-Fraction Estimates are Accurate on a Validation Set and No Outliers are Observed Compared to ichorCNA

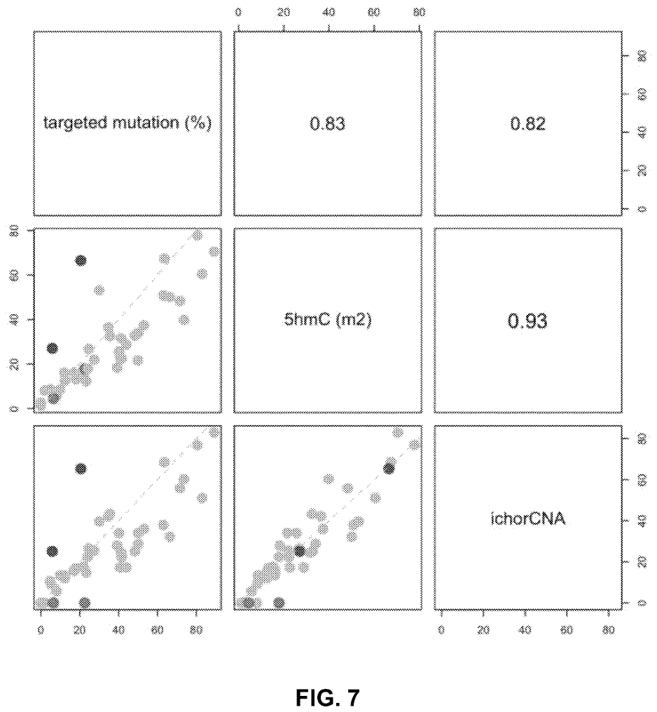

An additional cohort of mCRPC cfDNA samples (n=43) were used to estimate the accuracy of the tumor fraction estimates. As shown in FIG. 7, m0, m1 and m2 5hmC models vs ichorCNA predictions were compared with the targeted mutation-based tumor fraction estimates. The m2 model performed best in cross-validation as well as in validation, with lowest composite absolute error. 5hmC-based estimates had higher Pearson correlation with ichorCNA-based estimates (lower right scatter plot). Correlation values are shown in the upper panels.

In some embodiments, outlier predictions were assessed where the prediction % was more than 3 times lower or higher than the targeted mutation, with at least a 5% differential. Referring again to FIG. 7, outliers generated by ichorCNA are highlighted in the lower left panel (2 red dots and 2 blue dots). All red and blue dots in the scatter plots of FIG. 7 correspond to these ichorCNA outliers.

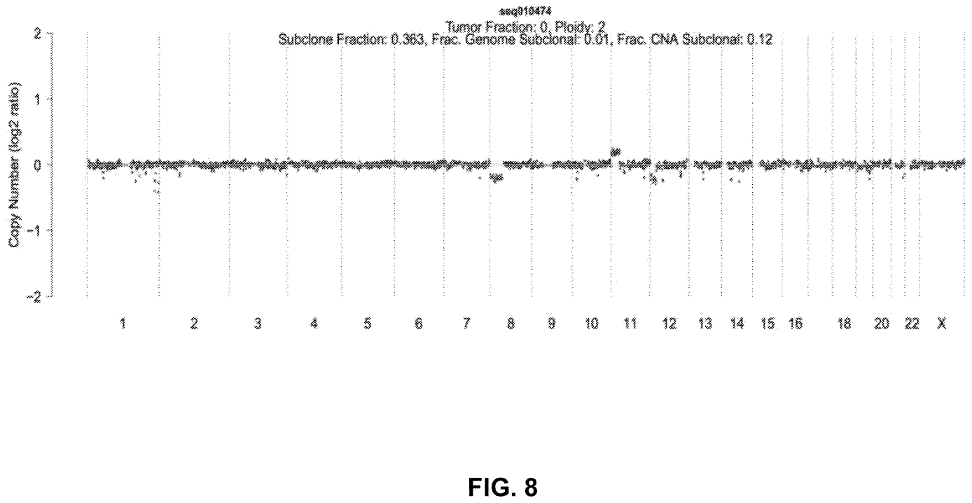

As shown, the 5hmC model does not generate the two lower-than-diagonal outliers (red) observed for ichorCNA but agreed with the above-the-diagonal outliers (upper left scatter plot). These two cases (in blue) are possible under-estimates of the mutation-based predictions. FIG. 8 shows the ichorCNA plot of the sample with 0% ichorCNA prediction, but ~25% targeted mutation and 5hmC predictions (red dot on middle bottom of ichorCNA facet).

### In Silico Simulations Achieve 90% Sensitivity and 97% Specificity at 3-20% Tumor Fraction Range

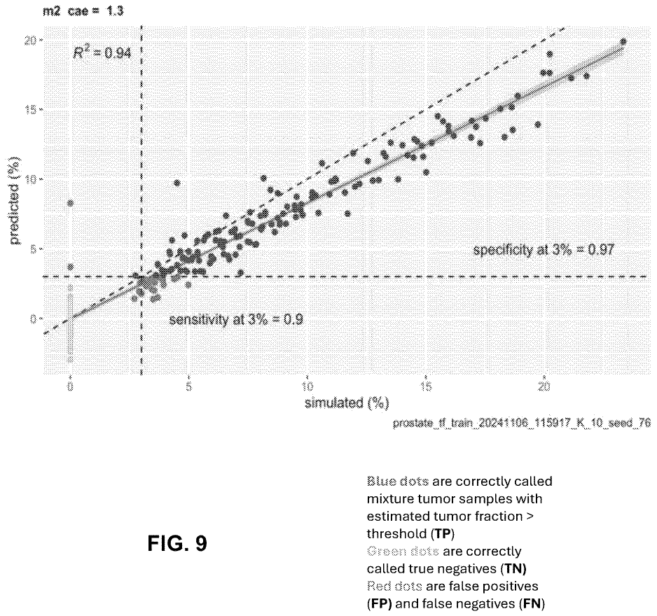

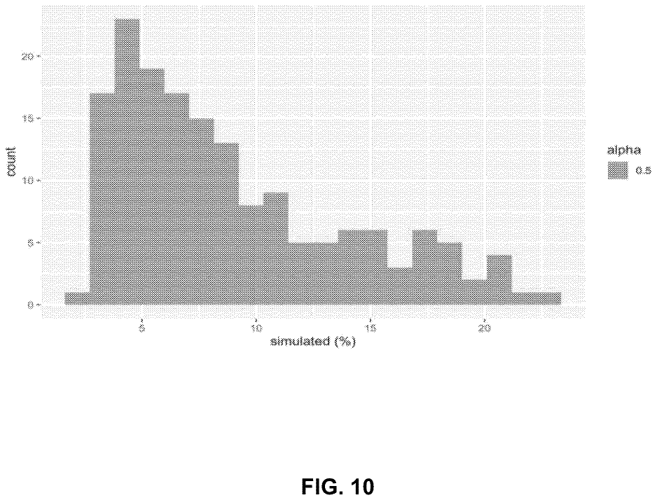

For each cancer validation sample with a predicted tumor fraction >10% (n=33), a mixture was made with 5 random non-cancer samples at slightly different ratios. Referring to FIG. 9, each mixture was de novo predicted by the model (y-axis) and the predictions were compared against the expected (%) that was calculated from 5hmC-based predictions of the components of the mixtures. The pool of non-cancer samples consisted of 74 samples. Ratios were selected to enrich for tumor fraction less than 10%, with expected (simulated %) tumor fractions shown in the histogram of FIG. 10.

### Tumor Fraction is Predictive of Survival

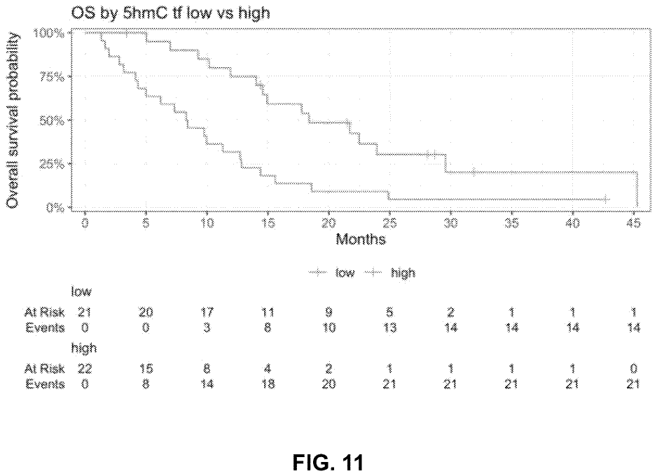

cfDNA samples were taken before first-line androgen receptor signaling inhibitor for mCRPC. Referring to FIG. 11, predicted 5hmC based tumor fraction estimates were stratified to two groups based on median tumor fraction estimate which was 22.3%. The Kaplan-Meier plot shown demonstrates the prognostic value of 5hmC-based ctDNA fractions through overall survival, indicating its potential role in predicting patient outcomes. The tumor fraction-high group had significantly lower overall survival (lower blue curve) compared to tumor fraction-low group (higher red curve) with a p-value of 0.001 (log-rank test).

Survival probability at 1-year was 75% and 30% respectively for low and high tumor fraction groups. Median (50%) survival was 18 months for TF-low and 8 months for TF-high group. The hazard ratio (HR) for this indicator with a cox-regression model was 2.8 (95% CI=1.60-4.90) with a p-value of 0.001. When controlling for age and visceral metastasis, the HR stayed significant at 2.92 (95% CI=1.66-5.14, p=0.0002).

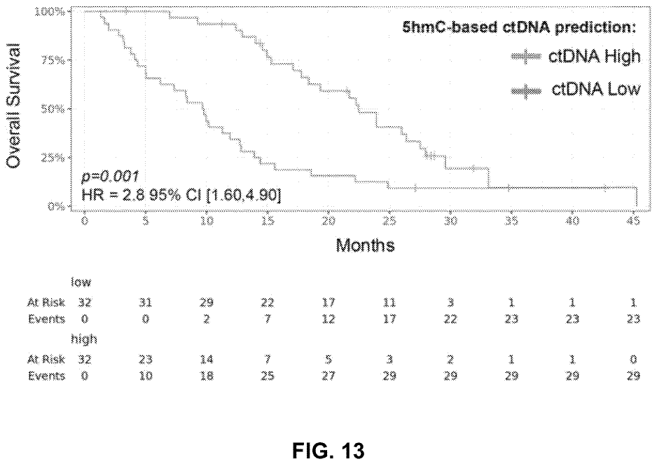

Referring to FIG. 13, a larger sample size was used to validate 5hmC based tumor fraction estimates as a prediction of survival. Predicted 5hmC based tumor fraction estimates were stratified to two groups based on a median tumor fraction estimate which was 18.0%. The sample included 64 samples with 32 samples lower and 32 samples higher than median cfDNA. The samples are denoted as ctDNA low (below median, red) and high (above median, blue). The ctDNA fraction-high group showed significantly lower overall survival (lower blue curve) compared to the ctDNA fraction-low group (higher red curve) with a HR of 2.8 (95% CI=1.60-4.90) with a p-value of 0.001.

Genes that are Positively and Negatively Associated with Tumor Fraction Form Distinct Clusters

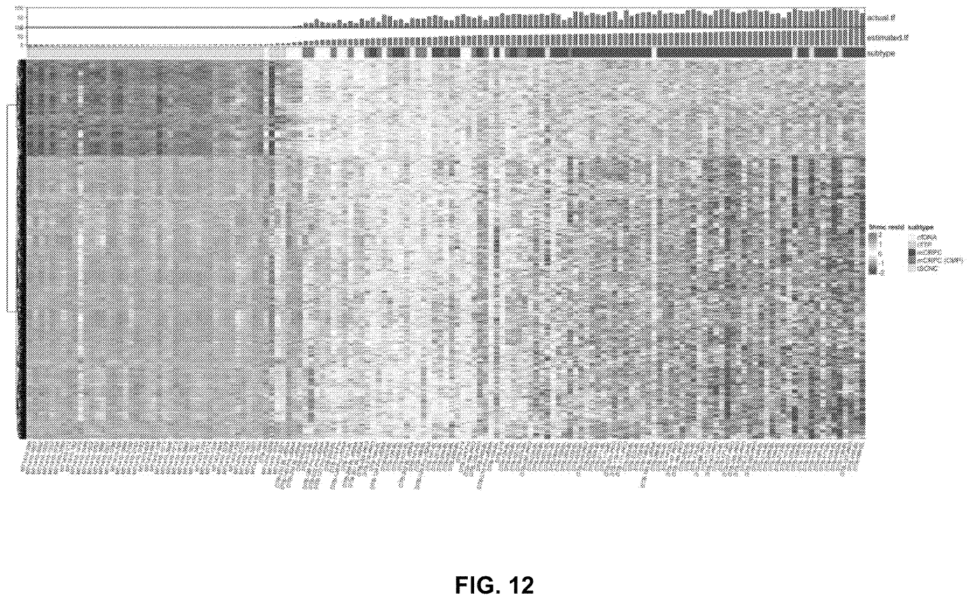

The top 1000 gene features based on their linear association with tumor fraction are displayed in FIG. 12. Genes are clustered based on their similarity. Two major clusters arise: a) genes that are positively associated with tumor fraction (top) and genes that are negatively associated with tumor fraction (bottom). Samples (columns) are sorted based on predicted tumor fraction.

Annotation column termed “actual.tf” is based on CNV estimates based on ichorCNA. The leftmost samples (green) are non-cancer and mostly have tumor fraction predictions of 0.

Biological Significance of Features Associated with Tumor Fraction

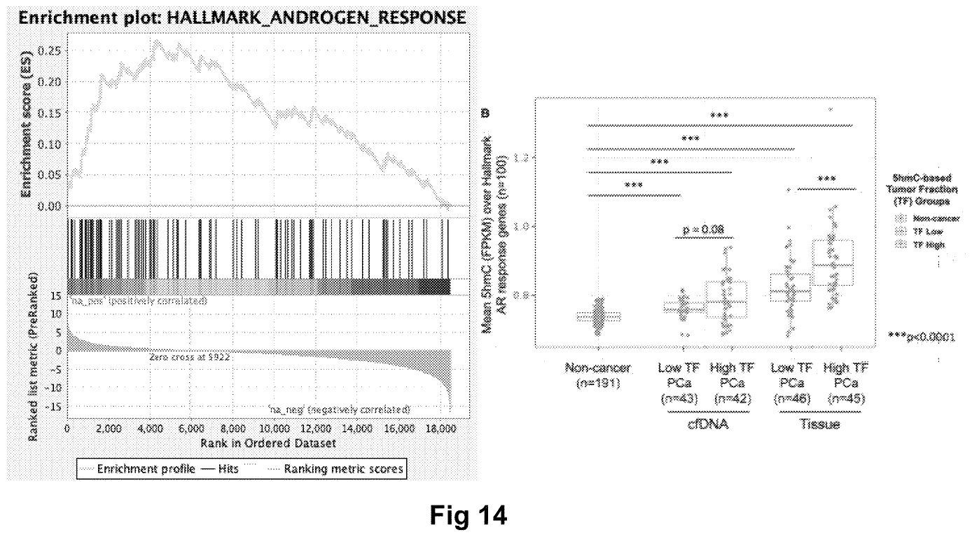

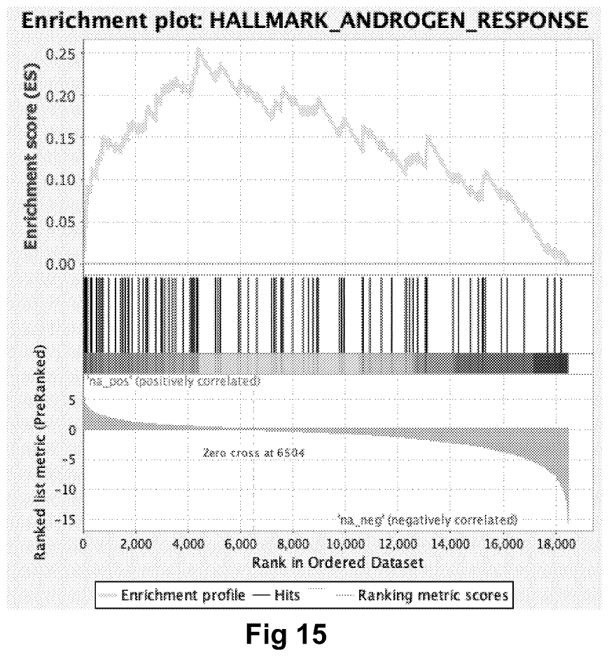

All 5hmC gene features' (after removal of CNV effect) linear association with tumor fraction p-value was input to Gene Set Enrichment Analysis (GSEA). p-values were augmented with their coefficient direction. FIG. 14 shows gene set enrichment analysis of 5hmC model features demonstrating enrichment of androgen response geneset and increased enrichment over androgen response geneset with increasing ct fraction. Using GSEA, Hallmark Androgen response genes marked with 5hmC were positively correlated with the tumor fraction. p-values from the linear association of 5hmC gene features with tumor fraction were used for GSEA. 5hmC features positively correlated with the tumor fraction show significant enrichment in the Hallmark Androgen response gene set. The zero cross was found to occur at rank 5922. A m0 rank order, shown in FIG. 15, found the zero cross at rank 6504.

Returning to FIG. 14, a boxplot shows significantly elevated 5hmC levels over Hallmark Androgen response genes in both high (above median) and low (below median) tumor fraction prostate cancer (PCa) samples compared to non-cancer controls. Median tumor fraction value for cfDNA and tissue samples was 24.5% and 62.9% respectively.

Positively associated hallmark gene sets (listed in Table 1) included androgen response. Negatively associated gene sets (listed in Table 2) were mostly associated with immune functions. This suggested a decrease in immune cell type signatures with increasing tumor fraction. Only significant gene sets are listed in the Tables below (FDR q-val<0.05).

TABLE 1
Gene sets positively associated with TF

| | GS | GS | | | | NO [symbol] | FDR |
|---|---|---|---|---|---|---|---|
| | follow link to MSlgDB | DETAILS | SIZE | ES | NES | p-val | q-val |
| 1 | HALLMARK ESTROGEN RESPONSE LATE | Details . . . | 199 | 0.22 | 3.43 | 0.000 | 0.000 |
| 2 | HALLMARK ESTROGEN RESPONSE EARLY | Details . . . | 200 | 0.19 | 3.12 | 0.000 | 0.000 |
| 3 | HALLMARK ANDROGEN RESPONSE | Details . . . | 100 | 0.27 | 3.06 | 0.000 | 0.000 |
| 4 | HALLMARK PANCREAS BETA CELLS | Details . . . | 40 | 0.29 | 2.19 | 0.006 | 0.011 |
| 5 | HALLMARK KRAS SIGNALING DN | Details . . . | 196 | 0.13 | 2.12 | 0.002 | 0.012 |
| 6 | HALLMARK CHOLESTEROL HOMEOSTASIS | Details . . . | 73 | 0.21 | 2.11 | 0.008 | 0.011 |
| 7 | HALLMARK MYC TARGETS V1 | Details . . . | 194 | 0.13 | 2.08 | 0.010 | 0.011 |
| 8 | HALLMARK G2M CHECKPOINT | Details . . . | 196 | 0.12 | 1.89 | 0.010 | 0.029 |
| 9 | HALLMARK SPERMATOGENESIS | Details . . . | 133 | 0.13 | 1.79 | 0.021 | 0.048 |

[symbol]

indicates data missing or illegible when filed

TABLE 2
Gene sets negatively associated with TF

| | GS | GS | | | | NO [symbol] | FDR |
|---|---|---|---|---|---|---|---|
| | follow link to MSlgDB | DETAILS | SIZE | ES | NES | p-val | q-val |
| 1 | HALLMARK INTERFERON GAMMA RESPONSE | Details . . . | 197 | −0.37 | −6.06 | 0.000 | 0.000 |
| 2 | HALLMARK ALLOGRAFT REJECTION | Details . . . | 199 | −0.34 | −5.46 | 0.000 | 0.000 |
| 3 | HALLMARK INTERFERON ALPHA RESPONSE | Details . . . | 95 | −0.41 | −4.83 | 0.000 | 0.000 |
| 4 | HALLMARK INFLAMMATORY RESPONSE | Details . . . | 199 | −0.24 | −3.98 | 0.000 | 0.000 |
| 5 | HALLMARK COMPLEMENT | Details . . . | 195 | −0.24 | −3.83 | 0.000 | 0.000 |
| | HALLMARK IL6 JAK STATS SIGNALING | Details . . . | 82 | −0.33 | −3.54 | 0.000 | 0.000 |
| | HALLMARK OXIDATIVE PHOSPHORYLATION | Details . . . | 197 | −0.19 | −3.04 | 0.000 | 0.000 |
| 8 | HALLMARK APOPTOSIS | Details . . . | 160 | −0.16 | −2.41 | 0.002 | 0.001 |
| 9 | HALLMARK TNFA SIGNALING VIA NEKB | Details . . . | 197 | −0.15 | −2.41 | 0.002 | 0.001 |
| 10 | HALLMARK IL2 STATS SIGNALING | Details . . . | 198 | −0.15 | −2.40 | 0.000 | 0.001 |
| 11 | HALLMARK DNA REPAIR | Details . . . | 147 | −0.16 | −2.32 | 0.002 | 0.002 |
| 12 | HALLMARK EPITHELIAL MESENCHYMAL TRANSITION | Details . . . | 198 | −0.11 | −1.88 | 0.006 | 0.022 |
| 13 | HALLMARK ADIPOGENESIS | Details . . . | 197 | −0.11 | −1.81 | 0.010 | 0.029 |
| 14 | HALLMARK HEME METABOLISM | Details . . . | 192 | −0.11 | −1.79 | 0.015 | 0.030 |

[symbol]

indicates data missing or illegible when filed

cfDNA-Derived 5hmC Profiles Distinguish AR-Driven and Neuroendocrine-Specific Prostate Cancer Subtypes

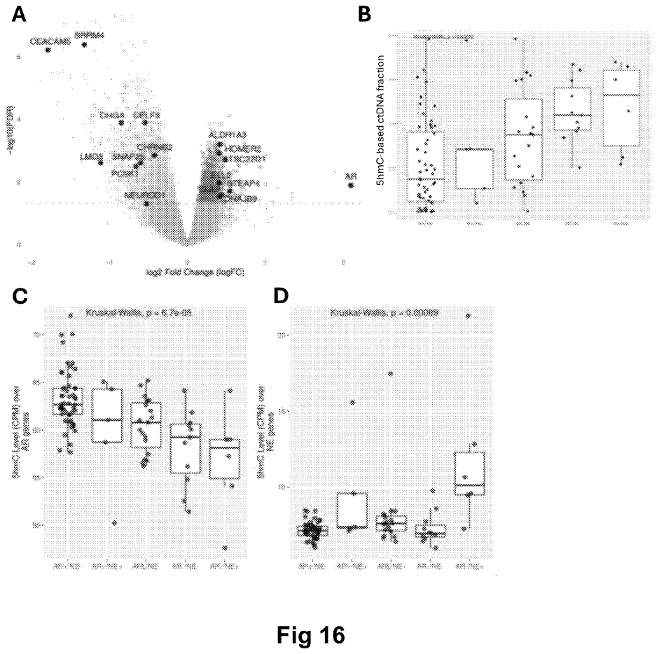

FIG. 16 shows plasma-derived cfDNA 5hmC profiles can distinguish between AR-specific and neuroendocrine-specific biology in prostate cancer according to one or more embodiments of the invention. A volcano plot is shown in the upper left, labelled A, of FIG. 16.

The volcano plot shows a differential 5hmC signal between AR-specific vs neuroendocrine-specific prostate cancer samples using cfDNA. The x-axis indicates log 2 fold change (log FC) and the y-axis shows −log 10 (FDR). Each point represents a gene. Genes enriched in the NE state are shown in yellow (negative log FC), and genes enriched in the AR state are shown in blue (positive log FC); nonsignificant genes are shown in grey. Markers for neuroendocrine-specific prostate cancer include CHGA, SYP, and NEURODL. Markers for the AR-specific lineage include AR and ALDH1A3. The dashed horizontal line denotes the FDR<0.05 threshold. In some embodiments, the markers include CEACAM5, SYP, SRRM4, AR, CHGA, CELF3, LMO3, SNAP25, CHRNB2, PCSK1, NEUROD1, ALDH1A3, HOMER2, TSC22D1, ELL2, STEAP4, TARP, and DNAJB9.

AR-specific and neuroendocrine-specific prostate cancer samples, defined by tissue RNA analysis, were used to assess differential cfDNA-derived 5hmC profiles between Androgen Receptor-active Prostate Cancer (ARPC) and Neuroendocrine Prostate Cancer (NEPC). Differential analysis identified subtype-defining genes among those with significantly altered 5hmC levels, with AR being the most enriched gene in ARPC relative to NEPC, while canonical neuroendocrine markers including NEUROD1 and CHGA were enriched in NEPC.

A distribution of 5hmC-based ctDNA fraction prediction in cfDNA samples is shown in the upper right of FIG. 16, labelled B. The samples include five prostate cancer subtypes listed in order from left to right of AR+/NE−, AR+/NE+, ARL/NE−, AR−/NE−, and AR−/NE+. The p-value was p=0.00072. Evaluation of 5hmC-based ctDNA fraction predictions across five prostate cancer subtypes (AR+/NE−, AR+/NE+, ARL/NE−, AR−/NE+, AR−/NE−) revealed significant lineage-associated differences. The ctDNA fraction increased progressively with reduced AR signaling, with AR+/NE− samples exhibiting the lowest ctDNA fractions and AR−/NE+ samples showing the highest levels. Elevated ctDNA fractions in AR−/NE+ tumors are consistent with NEPC biology, an aggressive and therapy-resistant phenotype characterized by high proliferative capacity, epigenetic reprogramming, and increased cell turnover.

The lower distributions of FIG. 16, labelled C and D respectively, depict boxplots showing plasma-derived cfDNA 5hmC levels (Counts Per Million, CPM) over AR genes (element C) and NE genes (element D) across five sample groups listed in order from left to right of AR+/NE−, AR+/NE+, ARL/NE−, AR−/NE−, and AR−/NE+. Individual points on the boxplots represent samples. Group differences were assessed using the Kruskal-Wallis test, with p-values shown above each panel.

Given the clear separation of ARPC and NEPC groups at the population level, 5hmC levels over AR response genes and NE-specific genes were examined across five different subtypes. 5hmC signal over AR response genes decreased quantitatively from AR-positive to NE-positive states, whereas NE-specific gene sets showed increased 5hmC enrichment in NE− positive subtypes relative to AR-positive groups. Together, these analyses demonstrate a coordinated and reciprocal shift in 5hmC landscapes between AR-associated and neuroendocrine transcriptional programs, highlighting the ability of cfDNA 5hmC profiling to robustly capture lineage-specific regulatory states and discriminate AR-drive from NE-like prostate cancer. The p-value for the AR response genes, element B of FIG. 16, was p=6.7e-05 and for the NE− specific genes, element C of FIG. 16, was p=0.00089.

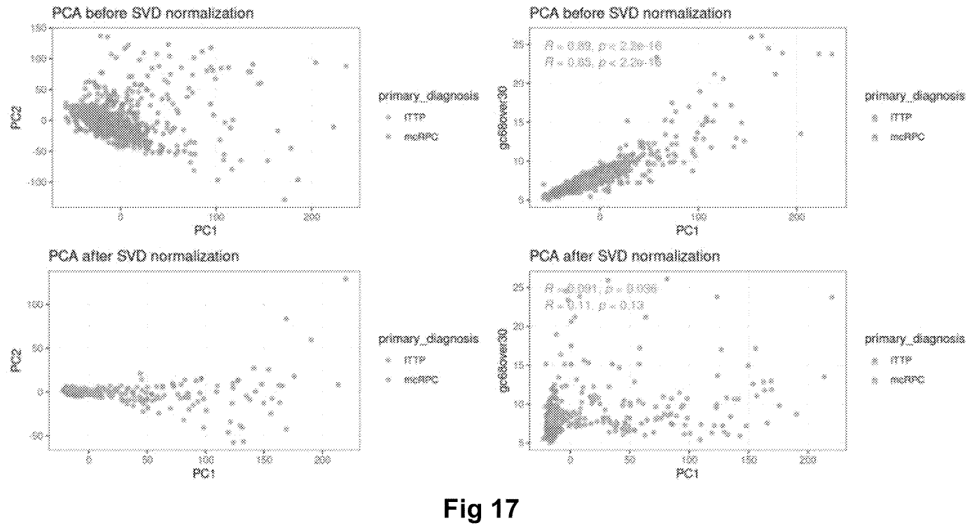

FIG. 17 shows SVD normalization reduces GC-dependent bias in 5hmC data according to one or more embodiments of the invention. A PCA plot using 5hmc genebody FPKM prior to SVD normalization is shown at the top left. At the top right, a scatter plot is shown illustrating PC1 vs gc68over30 (gc-bias metrics) found the Pearson correlation coefficient of ITTP (non-cancer) and mCRPC are both strong and significant. The non-cancer samples, ITTP, had a Pearson correlation coefficient of R=0.89 and a p-value of less than 2.2e-16. The cancer samples, mCRPC, had a Pearson correlation coefficient of R=0.85 and a p-value of less than 2.2e-16. The bottom left PCA plot shows 5hmC genebody FPKM following SVD normalization. At the bottom right, a scatter plot of PC1 vs gc68over30 following SVD normalization shows the Pearson correlation coefficient is reduced and p-values are much less significant. The non-cancer samples, ITTP, had a Pearson correlation coefficient of R=0.091 and a p-value of 0.036. The cancer samples, mCRPC, had a Pearson correlation coefficient of R=0.11 and a p-value of 0.13.

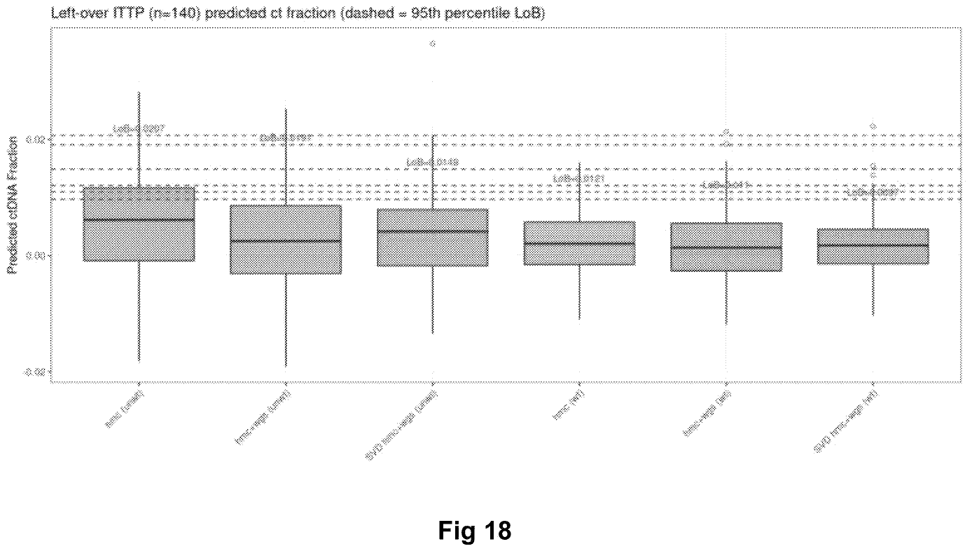

FIG. 18 shows a Limit of blank (LoB) reduction by adding WGS fragmentation features, SVD normalization and weighting in glmnet modeling according to one or more embodiments of the invention. In some embodiments, glmnet models were trained using cfDNA samples of mCRPC and non-cancer samples with features of: 5hmC genebody FPKM, 5hmC genebody FPKM and WGS fragmentation features, and SVD normalized 5hmC genebody FPKM and WGS fragmentation features. The models were trained either weighted or unweighted. In an embodiment, the weight scheme may include a sample-specific weighting scheme incorporated into the glmnet optimization framework where each observation contributes to a penalized loss proportional to an assigned weight, enabling differential influence on model fitting. A Limit of Blank (LoB) is a threshold that 95% of non-cancer (ITTP) samples have predicted ct fraction values below the cutoff. The boxplots show predicted ct fractions for a sample of 140 non-cancer samples that were not part of a training set for each specified feature set, SVD normalization or weighted or not, with horizontal lines illustrating the LoB for each model. The LoB for each model, going left to right is: hmc unweighted=0.0207, hmc+WGS unweighted=0.0191, SVD normalized hmc+WGS unweighted=0.0149, hmc weighted=0.0121, hmc+WGS weighted=0.011, and SVD normalized hmc+WGS weighted=0.0097.

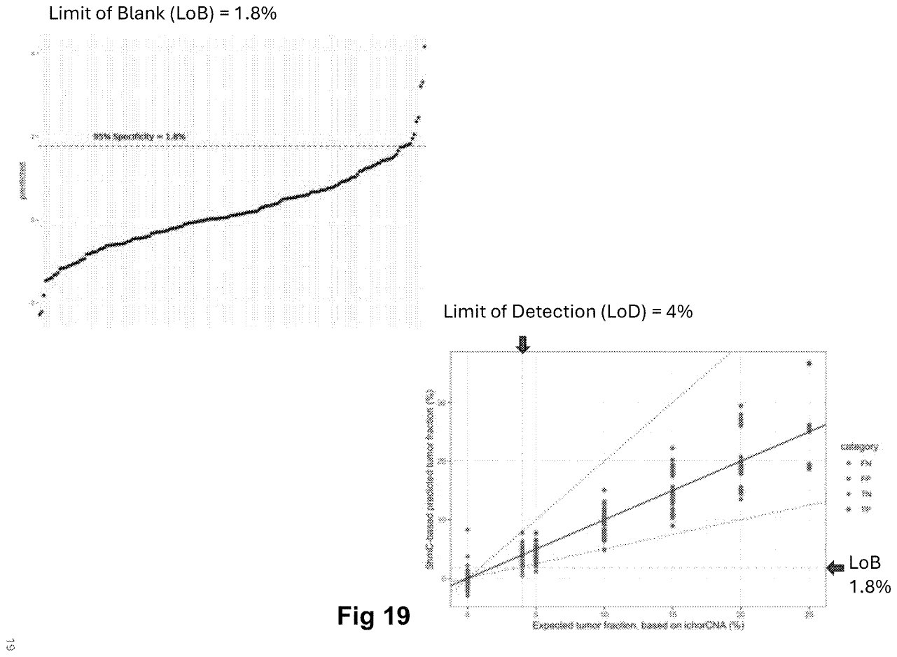

FIG. 19 shows a Limit of Blank (LoB) and Limit of Detection (LoD) of an embodiment of a model for estimating ct fraction according to the disclosure. In an embodiment, the LoB was 95% specificity of non-cancer cfDNA samples (collected in streck tubes) with a predicted ct fraction. The samples used were independent of a training data set. The LoD used an in-silico dilution of 5 prostate cfDNA cancer into randomly selected 10 out of 74 non-cancer with target ct fraction at 0.05, 0.1, 0.5, 1, 2, 3, 4, 5, 10, 15, 20, 25. The LoD was calculated at a given ct fraction such that >=95% of samples are detected, i.e. >LoB cutoff.

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A method of estimating a tumor fraction in a sample, the method comprising:
separating a first portion of cfDNA from a biological sample of cfDNA fragments containing epigenetic markers;
sequencing the separated first portion of cfDNA to generate a first set of genomic sequences;
sequencing cfDNA remaining in the sample after the separating step to generate a second set of genomic sequences;
mapping the first and second set of genomic sequences to reference sequences;
normalizing counts of mapped sequences to generate normalized first genomic sequence counts and second genomic sequence counts;
identifying repeated genomic insertions and/or deletions by measuring a depth of sequence read coverage from the second set of genomic sequences;
subtracting the repeated insertions and/or deletions identified in the identifying step from the first set of genomic sequences to determine a number of residual genomic sequence counts; and
estimating the tumor fraction as a fraction of the cfDNA that is tumor derived based on the number of residual and/or normalized genomic sequence counts.

2. The method of claim 1, wherein estimating the tumor fraction comprises operating a machine learning system trained on sequence data from at least tumor tissue/cfDNA, and healthy tissue/cfDNA.

3. The method of claim 1, wherein the normalized read counts comprise fragments per kilobase per million fragments (FPKM).

4. The method of claim 3, wherein the FPKM are log-transformed.

5. The method of claim 3, wherein the input to the machine learning system comprises concatenation of residuals, the FPKM values, fragmentomics or other fragment properties.

6. The method of claim 2, wherein the machine learning system comprises a regularized linear regression classifier, and other non-linear classifiers.

7. The method of claim 2, further comprising building the machine learning system by:
sequencing nucleic acid from samples with known tumor fractions to obtain genomic reads;
treating and sequencing portions of the nucleic acid comprising modified bases to obtain modified reads;
mapping the genomic reads and the modified reads to reference data;
normalizing counts of mapped reads to provide normalized genomic read counts and normalized modified read counts;
removing effects of copy number variation (CNV) from the normalized genomic read counts and saving the result as a residual, wherein the residual comprises normalized modified read counts corrected to remove the effects of the CNV; and
training the machine learning system using at least the residual, the normalized genomic read counts, and the known tumor fractions to output an estimated tumor fraction when given the input comprising at least the subject residual or normalized genomic read counts.

8. The method of claim 2 wherein the cancer is selected from breast, colorectal, gastric, prostate, esophagus, liver, lung, ovarian and pancreatic cancer.

9. The method of claim 1, further comprising normalizing at least the first genomic sequence count using SVD methodology.

10. The method of claim 1, wherein the first genomic sequence count and second genomic sequence count are normalized using SVD normalization.

11. The method of claim 1, wherein the epigenetic marker is 5-hydroxymethylcytosine (5hmC).

12. The method of claim 1, wherein the epigenetic markers include a marker selected from the group consisting of: 5hmC, CEACAM5, SYP, SRRM4, AR, CHGA, CELF3, LMO3, SNAP25, CHRNB2, PCSK1, NEUROD1, ALDH1A3, HOMER2, TSC22D1, ELL2, STEAP4, TARP, and DNAJB9.

13. A method of estimating a tumor fraction in a sample, the method comprising:
separating a portion of cfDNA from a biological sample of cfDNA fragments containing epigenetic markers;
sequencing the separated portion of cfDNA to generate a sequence library;
mapping the sequence library to a reference sequence;
normalizing counts of mapped sequences to generate a normalized genomic sequence count;
identifying repeated genomic insertions and/or deletions by measuring a depth of sequence read coverage;
subtracting the repeated insertions and/or deletions identified in the identifying step from the sequence library to determine a number of residual genomic sequence counts; and
estimating the tumor fraction as a fraction of the cfDNA that is tumor derived based on the number of residual and/or normalized genomic sequence counts.

14. The method of claim 13, wherein estimating the tumor fraction comprises operating a machine learning system trained on sequence data from at least tumor tissue/cfDNA, and healthy tissue/cfDNA.

15. The method of claim 13, wherein the normalized read count comprises fragments per kilobase per million fragments (FPKM).

16. The method of claim 15, wherein the FPKM are log-transformed.

17. The method of claim 15, wherein the input to the machine learning system comprises concatenation of residuals, the FPKM values, fragmentomics or other fragment properties.

18. The method of claim 14, wherein the machine learning system comprises a regularized linear regression classifier, and other non-linear classifiers.

19. The method of claim 14, further comprising building the machine learning system by:
sequencing nucleic acid from samples with known tumor fractions to obtain genomic reads;
treating and sequencing portions of the nucleic acid comprising modified bases to obtain modified reads;
mapping the genomic reads and the modified reads to reference data;
normalizing counts of mapped reads to provide normalized genomic read counts and normalized modified read counts;
removing effects of copy number variation (CNV) from the normalized genomic read counts and saving the result as a residual, wherein the residual comprises normalized modified read counts corrected to remove the effects of the CNV; and
training the machine learning system using at least the residual, the normalized genomic read counts, and the known tumor fractions to output an estimated tumor fraction when given the input comprising at least the subject residual or normalized genomic read counts.

20. The method of claim 14 wherein the cancer is selected from breast, colorectal, gastric, prostate, esophagus, liver, lung, ovarian and pancreatic cancer.
